(19) United States
(12) Patent Application Publication
(10) Pub. No.: US 2009/0126736 A1
(43) Pub. Date: May 21, 2009

(54) In-home medical data collection and reporting system

(76) Inventors: Brenton Taylor, Kenwood, CA (US); Geoffrey Deane, Bellevue, WA (US)

Correspondence Address: MARK RODGERS, 1590 SAN ROQUE ROAD, SANTA BARBARA, CA, 93105, US

(21) Appl. No.: 12/218,036

(22) Filed: Jul. 10, 2008

Related U.S. Application Data
(60) Provisional application No. 60/959,690, filed on Jul. 16, 2007.

(51) Int. Cl. A61M 16/00; A61B 5/00

(52) U.S. Cl. 128/204.23; 600/301

(57) ABSTRACT

The invention is an oxygen system, including a home care system, for patients who require supplemental oxygen. Built around an intelligent portable oxygen concentrator, the system incorporates a variety of patient monitoring and reporting function enabled by the processing and communications channels built into the concentrator.

### RELATED APPLICATIONS

This Application claims priority to U.S. Provisional Application Ser. No. 60/959,690, filed Jul. 16, 2007

### FEDERALLY SPONSORED RESEARCH

Not Applicable

### SEQUENCE LISTING

Not Applicable

### BACKGROUND OF THE INVENTION

The invention relates to in-home or portable medical data collection and reporting, and in particular to in-home situations where an intelligent oxygen concentrator is present. The application is particularly directed to a system which includes a modern, portable oxygen concentrator.

The application of oxygen concentrators for therapeutic use is known, and many variants of such devices exist. A particularly useful class of oxygen concentrators is designed to be portable, allowing users to move about and to travel for extended periods of time without the need to carry a supply of stored oxygen. Most of these portable concentrators are based on Pressure Swing Adsorption (PSA) or Vacuum Pressure Swing Adsorption (VPSA) designs which feed compressed air to selective adsorption beds. In a typical oxygen concentrator, the beds selectively adsorb nitrogen, resulting in pressurized, oxygen-rich product gas.

The main elements in an oxygen concentrator are shown in FIG. 1. Air is drawn in, and typically filtered, at air inlet 1 before being pressurized by compressor 2. The pressurized air is directed by a valve arrangement through adsorbent beds 3. An exemplary adsorbent bed implementation, used in a concentrator design developed by the inventors, is three columns filled with zeolite powder. The pressurized air is directed through these columns in a series of steps which constitute a PSA cycle. Although many different arrangements of beds are possible as well as a variety of different PSA cycles, the result is that nitrogen is removed by the adsorbent material, and the resulting oxygen rich air is routed to a product gas storage device at 4. Some of the oxygen rich air is routed back through the bed to flush out (purge) the trapped nitrogen to an exhaust. Generally multiple beds, or columns in the exemplary device, are used so at least one bed may be used to make product while at least one other is being purged, ensuring a continuous flow of product gas. The purged gas is exhausted from the concentrator at 6.

Such PSA systems are known in the art, and it is appreciated that the gas flow control through the compressor and the beds in a PSA cycle is complex and requires precise timing and control of parameters such as pressure, flow rate, and temperature to attain the desired oxygen concentration in the product gas stream. Accordingly, most modern concentrators also have a programmable controller 2, typically a microprocessor, to monitor and control the details of the PSA cycle and monitor various parameters. Typically, due to the availability of inexpensive processor hardware, the controller can be configured to have significant processing and communications capability in excess of that required to run the concentrator, with no significant cost penalty. Thus the presence of an in-home concentrator provides the possibility of significant functionality which could be applied to patient and caregiver needs.

Patients who require in-home oxygen generally need medical monitoring of other vital parameters, such as blood oxygen saturation, blood pressure, body temperature and the like. Currently, either the patient must visit a medical facility or be visited in the home by a technician to gather such information. This is both costly and inconvenient. Moreover, under these conditions, the patient monitoring may not happen frequently enough to be effective.

Thus there is clear need for a system that would provide convenient, frequent in-home patient monitoring, particularly for patients requiring supplemental oxygen.

Such a system could reduce the overall burden on the healthcare system by alerting clinicians to potential changes in health status before the health of the patient reaches the level where emergency intervention or hospitalization is required to end an acute episode.

Similarly, such a monitoring system would alert the Home Medical Equipment Provider to a potential malfunction of the device prior to the device failing and requiring an unscheduled replacement or trip to the patient's home, thus reducing the cost burden on the HME.

### BRIEF SUMMARY OF THE INVENTION

The invention is a system and a process for the use thereof, which includes a portable oxygen concentrator, including a programmable controller and a communications channel coupled to the controller, patient monitoring devices including at least one of a pulse oximeter, a blood pressure monitor, a temperature monitor, electronic scale, body composition analyzer or a spirometer, such that the devices interface to the controller, and a program application running on the controller adapted to prompt a patient to use one or more of the monitoring devices on a predetermined basis and to report both monitoring device results and concentrator use data over the communications channel.

In a preferred embodiment, the communications channel may be one or more wireless devices, chosen from a group including IrDA, cell phone interfaces, blue tooth interfaces, Wi-fi, Zigbee, or dedicated radios. The communications channel preferably accesses the internet directly or through a secondary communication device to provide the data report.

In another embodiment, the system also includes a program application adapted to monitor and report patient activity. One measure of patient activity is battery usage of the concentrator. Another measure of patient activity is feedback from a motion sensor, Global Positioning Device, or accelerometer.

In other embodiments, the system may include speakers for audible alerts and status, as well as microphones to accept audible commands. One such command, for the case where the system has a cellular interface is to accept an audible command to connect to 911. Also a microphone can be used to sample the audio environment, and the controller may generate a noise cancellation signal which may be output through a speaker.

In another embodiment, the system may include a GPS unit and the GPS data may be part of the reported information and a part of the emergency response feature.

### BRIEF DESCRIPTION OF THE DRAWINGS

The understanding of the following detailed description of the invention will be facilitated by referring to the accompanying figures.

FIG. 1 shows the general elements of gas concentrators as applicable to the invention.

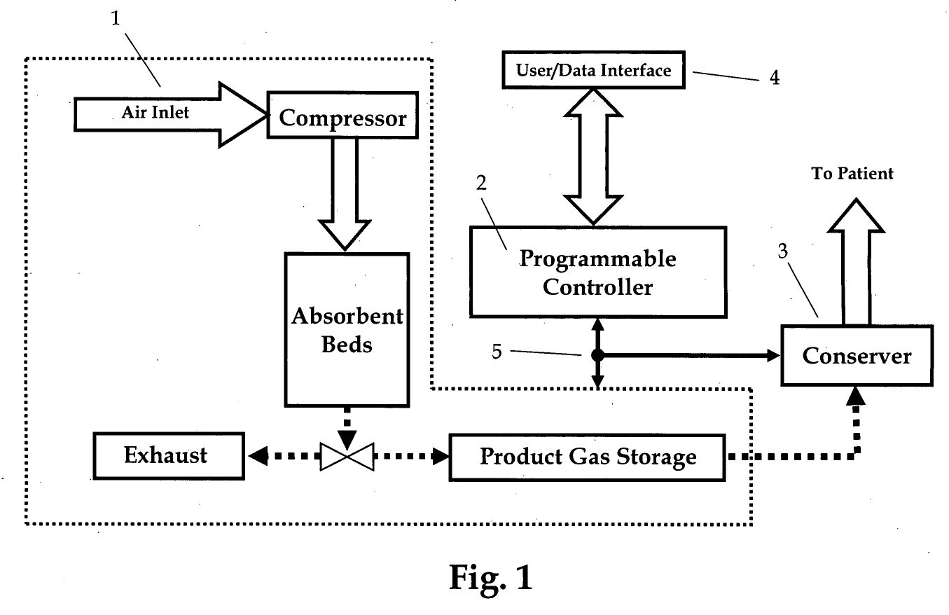

FIG. 2 illustrates the general operation of the invention.

FIG. 3 is a block diagram of an embodiment of the invention.

FIG. 4 is a block diagram of another embodiment of the invention.

### DETAILED DESCRIPTION OF THE INVENTION

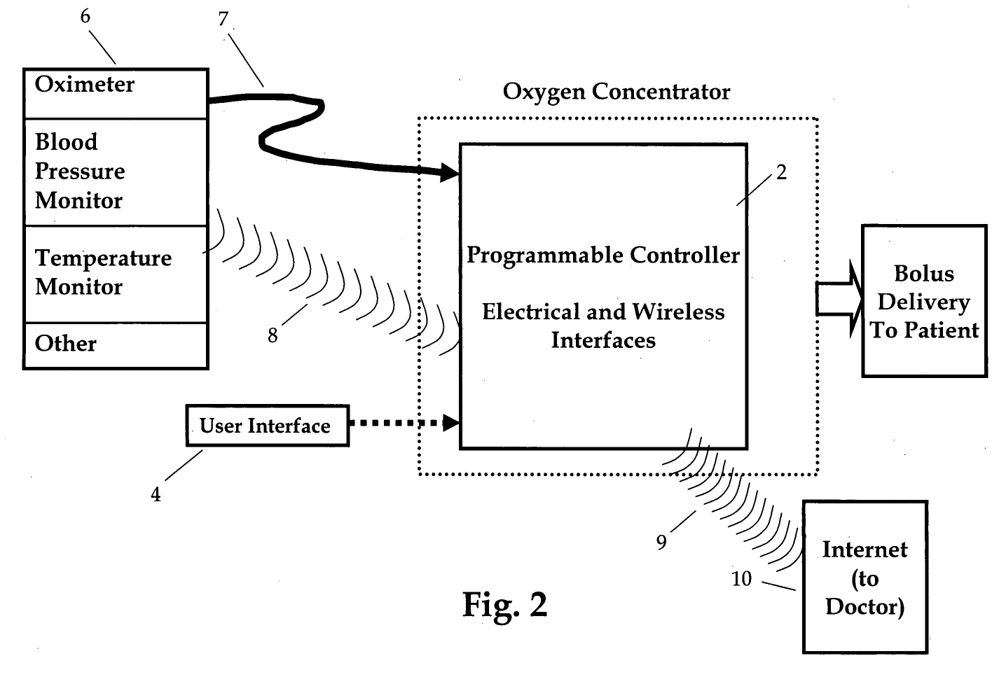

Referring to FIG. 2, an embodiment of the novel system is illustrated. The central element is the oxygen concentrator, and in particular the programmable controller 2. The programmable controller is interfaced to one or more of a suite of medical monitoring devices 6. These devices may include a pulse oximeter, blood pressure monitor, temperature monitor, or other less common devices, such as spirometers. The interface may be through a wired connection 7, either through general purpose interfaces such as USB or dedicated device-specific interfaces if required. One skilled in the art will readily comprehend a variety of suitable electrical interface panels and the like. Preferably, devices would be used that connect to the controller wirelessly 8 as such an arrangement would be more convenient for the patient. The Bluetooth or Zigbee standard is an example of suitable wireless system for connecting appliances to a controller.

The controller will contain a software application to acquire data from the suite of medical monitors. This application preferably also performs other functions. A particularly useful function would be to remind the patient to use the monitors at appropriate times and intervals, possibly using the concentrator's user interface 4 or a dedicated interface system auxiliary to the concentrator.

In the preferred embodiment, the controller will have a communications channel 9 to the outside world, and will use this channel to communicate with the patient's doctor or caregiver. This interface could be a variety of wired or wireless interfaces. However in the preferred embodiment, the connection is to the internet 10. Connection to the Internet facilitates a web-page approach to presenting patient information to the Doctor. Such an approach is particularly convenient both in terms of flexibility of data management and presentation, as well as providing universal access from a wide variety of locations and connection devices, i.e. office computers, PDA's, laptops, cell phones etc, allowing for convenient patient monitoring at any time or location.

Using the Internet as the data presentation medium also allows for novel business practices, as described in a co-pending application by the same inventors.

Other patient data may be gathered from the use of the concentrator itself, which through the programmable controller is capable of a fair amount of patient monitoring due to its own operation. For instance patient activity may be inferred by battery usage of the concentrator or the output of a motion sensor or accelerometer, indicating how much moving away from a fixed power source is taking place or the general activity level of the patient. Such information may be logged by the controller as provided along with the other data to the remote caregiver. Additionally, high levels of acceleration may signal that the device has been dropped or abused, and some device inspection or inquiry may be required.

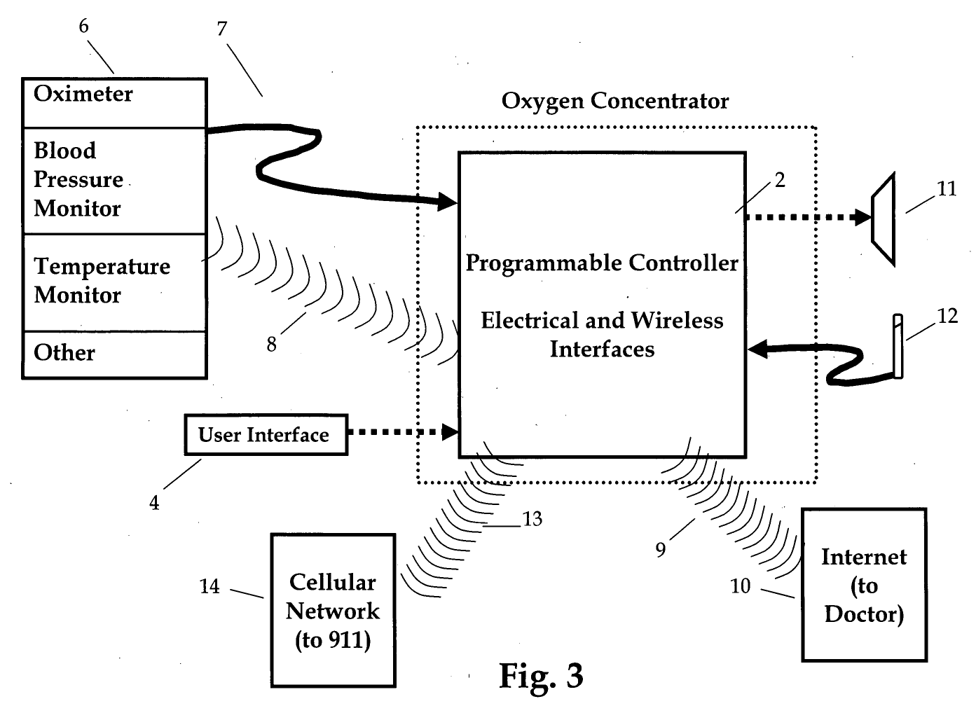

Referring to FIG. 3, other versions of the system are illustrated. The system may include a speaker and driver 11, interfaced to controller 2. The speaker 11 may be used for audible alerts, reminders, or device status messages. A microphone 12 may also be interfaced to the controller. The microphone driver, may be configured for speech recognition, allowing for patient commands to be provided audibly, allowing for less moving around by the patient. If the system is a version that includes a cellular network interface 14, the patient could connect to 911 (or a caregiver) without moving as long the patient was in range of the microphone.

Another useful aspect of the system possible for versions with both speaker and microphone is active noise cancellation (ANC). A controller application could sample the ambient audio environment using the microphone 12 and compute and generate a cancellation signal which could be output through the speaker 11. Such a feature could be quite effective at improving the patient environment, particularly such as reducing apparent compressor or fan noise which is generally a byproduct of the concentrator operation.

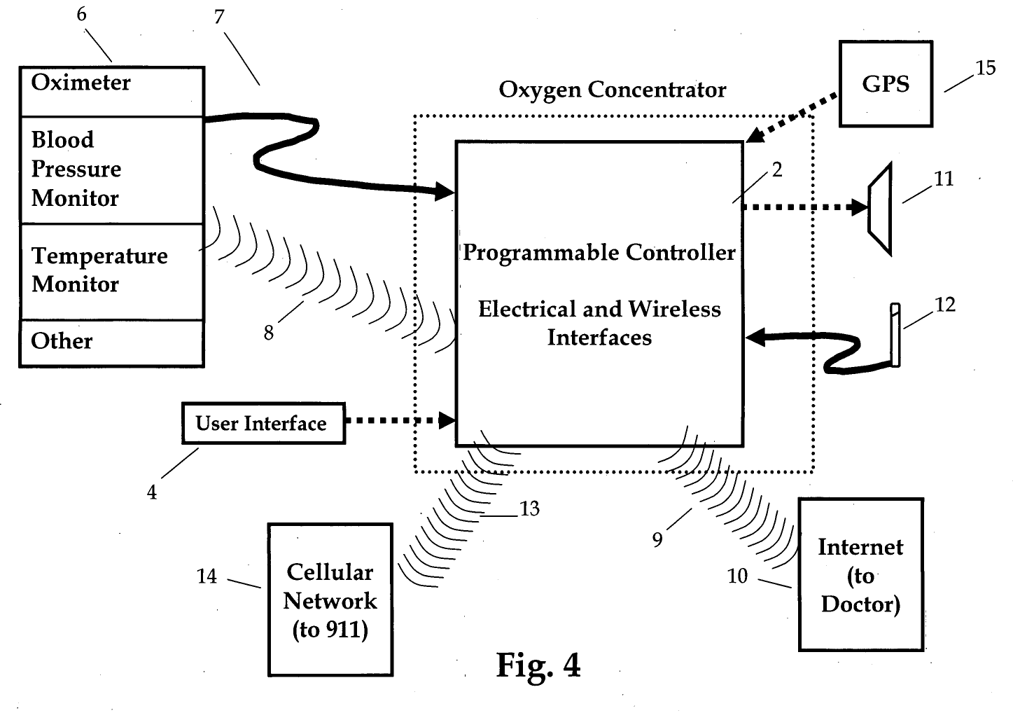

In FIG. 4 another version of the system is shown which includes a GPS unit 15. With such a unit attached, the system could also report patient position, either to the caregiver over a network, or by radio or cell to emergency personnel. The GPS unit can also be used for inventory tracking—if the device has a cell phone feature built in, the cell phone can be called to find the device, provided power is available to the concentrator. Since a significant number of devices get lost when patients expire, such a feature could serve to lower the overall cost of care.

We claim:

1. A supplemental oxygen care system comprising;
a portable oxygen concentrator, including a programmable controller and a communications channel coupled to the controller,
patient monitoring devices including at least one of a pulse oximeter, a blood pressure monitor, a temperature monitor, electronic scale, body composition analyzer or a spirometer, wherein the device are coupled to the controller; and,
a program application running on the controller adapted to prompt a patient to use one or more of the monitoring devices on a predetermined basis and to report both monitoring device results and concentrator use data over the communications channel.

2. The system of claim 1 wherein the communications channel comprises one or more wireless devices, chosen from a group including cell phone interfaces, blue tooth interfaces, Wi-Fi, Zigbee, or dedicated radios.

3. The system of claim 1 wherein the communications channel accesses the internet to provide the data report.

4. The system of claim 1 further comprising a program application adapted to monitor and report patient activity.

5. The system of claim 4 wherein the program application uses at least one of battery usage or a signal from a motion detector as the parameter to determine patient activity.

6. The system of claim 1 further comprising;
a speaker system coupled to the controller; and
a program application adapted to provide audible indications of system status and settings in the form of words and messages in combination with typical alarms or buzzers.

7. The system of claim 1 further comprising;
a microphone coupled to the controller; and,
a program application adapted to accept audible commands.

8. The system of claim 2 further comprising;
a microphone coupled to the controller; and,
a program application adapted to accept audible commands.

9. The system of claim 6 further comprising;
a microphone coupled to the controller; and,
a program application adapted to accept audible commands.

10. The system of claim 8 wherein the program application is further adapted to use the cell phone channel to dial 911 in response to the appropriate audible command or button press.

11. The system of claim 2 further comprising;
a microphone coupled to the controller; and,
a driver to utilize microphone data to determine and output a noise cancellation signal to the at least one speaker.

12. The system of claim 1 further comprising a GPS module, wherein GPS data may also be reported over the communications channel.

13. A supplemental oxygen care method, comprising;
interfacing patient monitoring devices including at least one of a pulse oximeter, a blood pressure monitor, a temperature monitor, electronic scale, body composition analyzer or a spirometer, to the programmable controller of a portable oxygen concentrator; and,
executing a program application running on the controller causing the controller to prompt a patient to use one or more of the monitoring devices on a predetermined basis and to report both monitoring device results and concentrator use data over a communications channel.

14. The method of claim 13 wherein the communications channel is one or more wireless devices, chosen from a group including IrDA, cell phone interfaces, blue tooth interfaces, Wi-FI, Zigbee, or dedicated radios.

15. The method of claim 13 wherein the communications channel accesses the internet to provide the data report.

16. The method of claim 13 further comprising executing a program application causing the controller to monitor and report patient activity.

17. The method of claim 16 wherein the program application uses at least one of battery usage or a signal from a motion detector as the parameter to determine patient activity.

18. The method of claim 13 further comprising;
coupling a speaker system to the controller; and
executing a program application which uses the speaker system to provide spoken indications of system status and settings.

19. The method of claim 13 further comprising;
coupling a microphone to the controller; and,
executing a program application which uses the microphone to accept audible commands.

20. The method of claim 14 further comprising;
coupling a microphone to the controller; and,
executing a program application which uses the microphone to accept audible command.

21. The method of claim 18 further comprising;
coupling a microphone to the controller; and,
executing a program application which uses the microphone to accept audible command.

22. The method of claim 20 wherein the program application further uses the cell phone channel to dial 911 in response to the appropriate audible command or button press.

23. The method of claim 14 further comprising;
coupling a microphone to the controller; and,
using a driver to utilize microphone data to determine and output a noise cancellation signal to the speaker.

24. The method of claim 13 further comprising utilizing a GPS module and a reporting GPS data over the communications channel.
